(19) United States
(12) Patent Application Publication
(10) Pub. No.: US 2024/0238032 A1
(43) Pub. Date: Jul. 18, 2024

(54) PLASMA PROBE FOR MEDICAL TREATMENT OF TISSUE

(71) Applicant: Erbe Elektromedizin GmbH, Tuebingen (DE)

(72) Inventors: Johannes HEYM, Tuebingen (DE); Martin WALZ, Rottenburg (DE)

(21) Appl. No.: 18/406,970

(22) Filed: Jan. 8, 2024

(30) Foreign Application Priority Data
Jan. 17, 2023 (EP) 23152010.7

(51) Int. Cl. A61B 18/04

(52) U.S. Cl. CPC A61B 18/042; A61B 2018/00083

(57) ABSTRACT

A flexible plasma probe having a head piece with at least one plasma exit window that is neither radially nor axially, but arranged in the tapering end section of the head piece with opening direction (O) orientated obliquely relative to the longitudinal center axis (L). The flexible plasma probe may simplify and improve treatment of biological tissue, particularly lesions in body lumens by allowing greater proximity between the electrode and the lesion.

This application claims priority to European Patent Application No. 23152010.7, filed Jan. 17, 2023, the entirety of which is incorporated by reference herein.

The invention refers to a plasma probe for treatment of biological tissue, particularly for the endoscopic use.

Plasma probes, particularly argon plasma probes, are frequently used for tissue coagulation or ablation. Different types of construction of such plasma probes are apparent from JP 2002-301088, for example. Accordingly, plasma probes are known having axial and/or lateral plasma exit openings. Particularly it is known to form such plasma probes from a flexible hose with a head piece distally attached thereon, which comprises an electrode supplied with HF-voltage. The head piece can be rounded at its distal end and can comprise lateral as well as an axial plasma exit opening.

GB 2 573 128 A discloses a plasma probe having a flexible hose and a spherically rounded end piece at its distal end. In the cylindrical section of the end piece lateral plasma exit openings are provided.

US 2013/0090644 A1 discloses a plasma probe having a hemispherically rounded end cap configured as sieve. Another instrument is disclosed by WO 2011/055368 A2.

In order to locate the plasma exit opening as close as possible to the tissue to be treated, EP 1 397 082 B1 discloses a plasma probe having a cylindrical basic structure and two lateral plasma exit openings that respectively follow a helical line. In the area of these plasma exit openings the probe body can be bent, so that upon putting the distal end of the probe onto the tissue and a respective lateral force application on the probe, a bending location is created at the plasma exit openings at which the plasma exit opening comes closer to the tissue compared with the elongated shape of the probe. This is particularly advantageous during treatment of pathological new tissue formations that project beyond other tissue, such as colon polyps or other lesions. However, this principle requires a flexibility of the probe in the area of the plasma exit opening, which can be a construction challenge.

Starting therefrom it is the object of the invention to provide a plasma probe of simplified construction type, which allows the plasma exit window to be brought close to the tissue to be treated.

This object is solved by a plasma probe according to claim 1:

An embodiment of a plasma probe according to the invention comprises a flexible hose that is provided with a rigid head piece at its distal end, which comprises an end section tapering in distal direction. The head piece comprises one or more plasma exit windows that extend at least partly into the tapering end section of the head piece. An electrode also extends into the head piece, wherein the electrode can be connected via an electrical conductor with an HF-generator and can be supplied by the latter with suitable HF-voltage. Gas, which is ionized by the electrode flowing through the hose forms a plasma flow that exits laterally to the longitudinal direction of the plasma probe from the plasma exit window transverse to the electrode. Because the plasma exit window is located in the tapering end section of the end piece or at least extends into this end section, tissue lesions can be specifically treated. The distance between the electrode and the tissue to be treated can be kept very small and concurrently the target location of the treatment can be defined very accurately. Preferably the taper of the head piece originates at the proximal end of the plasma exit window or plasma exit windows.

The plasma probe is preferably configured in monopolar manner, i.e. the plasma probe comprises only one single electrode and the current flows from the electrode to the tissue to be treated and therefrom via a neutral electrode attached to the patient back to the generator. Because the distance between the electrode and the tissue is minimized due to the design according to the invention, not only the influence location of the plasma jet can be defined precisely, but it can also be operated and the tissue can be influenced with comparable low power in order to not cause tissue damage beyond the necessary degree.

An embodiment of the plasma probe according to the invention is particularly suitable for use in narrow hollow vessels in which the probe is guided substantially parallel to the extension of the body lumen, e.g. by an endoscope, and only the end of the endoscope and the plasma probe is slightly angled toward the location to be treated. The rounded head piece thereby allows that the distal end of the probe slides on the tissue to be treated and thus functions like a skid. This applies particularly, if the tapering end section is not only hemispherically rounded, but in addition is configured slightly longitudinally rounded. Preferably the end piece comprises in the longitudinal section a rounded contour without corners. The longitudinal section can have the shape of a blunt rounded cone, i.e. a cone with rounded tip or also rounded flanks so that its surface lines extend in an arc-shaped manner. Indeed with this shape a particularly good effect is obtained.

Preferably the plasma probe comprises exclusively lateral plasma exit windows with plasma exit direction orientated transverse, but thereby slightly obliquely to the longitudinal direction of the plasma probe. Preferably the plasma probe according to the invention is thereby closed at the distal end. This means, no plasma exit opening with axial exit direction is provided. Between the plasma exit windows separating webs are arranged extending in axial direction. Measured in circumferential direction the sum of the width of the plasma exit windows is longer, preferably remarkably longer, than the sum of the widths of the webs provided between the plasma exit windows.

The head piece comprises preferably multiple plasma exit windows distanced in circumferential direction from one another at the same axial position, which are unexceptionally at least partly arranged in the tapering end section. Further preferably, only one single annulus of plasma exit windows is provided in the head piece, i.e. all plasma exit windows are arranged at the same axial position.

The electrode can be fixated in the hose and preferably fixated in the head piece and, for example, can be anchored for this purpose in the head piece at the distal end thereof. In another embodiment preferred at present the end of the electrode is, however, uncovered so that no direct thermal contact between the electrode and the insulation material of the head piece is provided. In doing so, the temperature of the head piece can be kept low on the outside in order to avoid sticking on the tissue.

The electrode can be held by a suitable holder, e.g. a sheet metal piece crossing the hose lumen inside the hose or in a tube-shaped extension of the head piece or in the hose as well as in the head piece. Thereby the electrode is centered in the proximity of the distal end of the plasma probe, so that the plasma probe can equally operate in all lateral directions.

The hose comprises at least a gas-conveying lumen that extends from its proximal end up to the head piece. Via a suitable connection on the proximal end of the hose, the lumen can be connected to a gas source and therefore allows a suitable gas, preferably argon, to flow longitudinally therethrough. The hose can alternatively comprise multiple gas conveying lumen that extend from the proximal end up to the distal end of the hose and commonly open out in the head piece.

Additional details of advantageous embodiments of the invention are derived from the drawings, the associated description or from the claims. In the drawings, embodiments of the invention are disclosed and show:

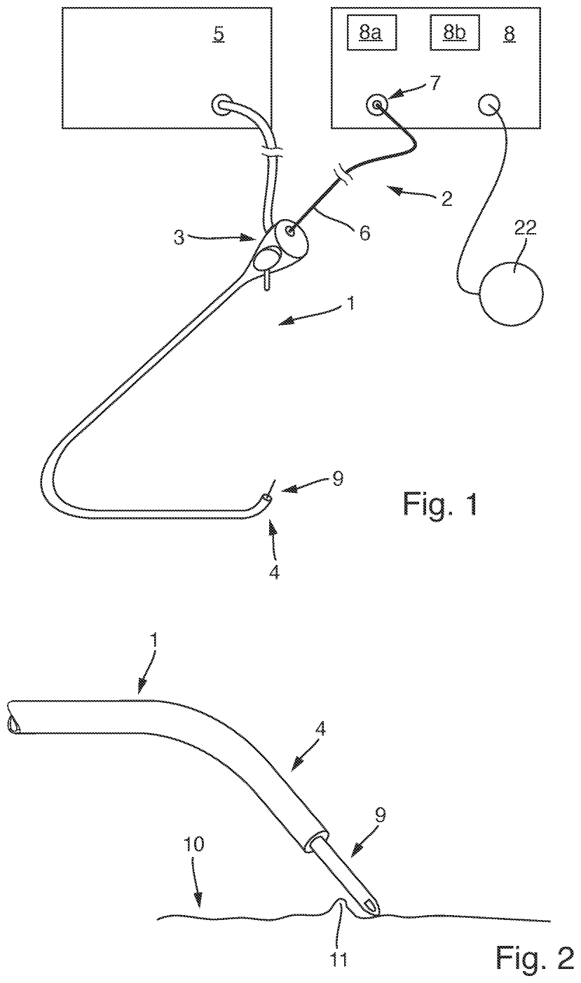

FIG. 1 illustrates an embodiment of an endoscope having a probe in accordance with the invention connected to supplying apparatuses in schematic illustration,

FIG. 2 illustrates an embodiment of a distal end of the endoscope and the probe during use on biological tissue in need of treatment,

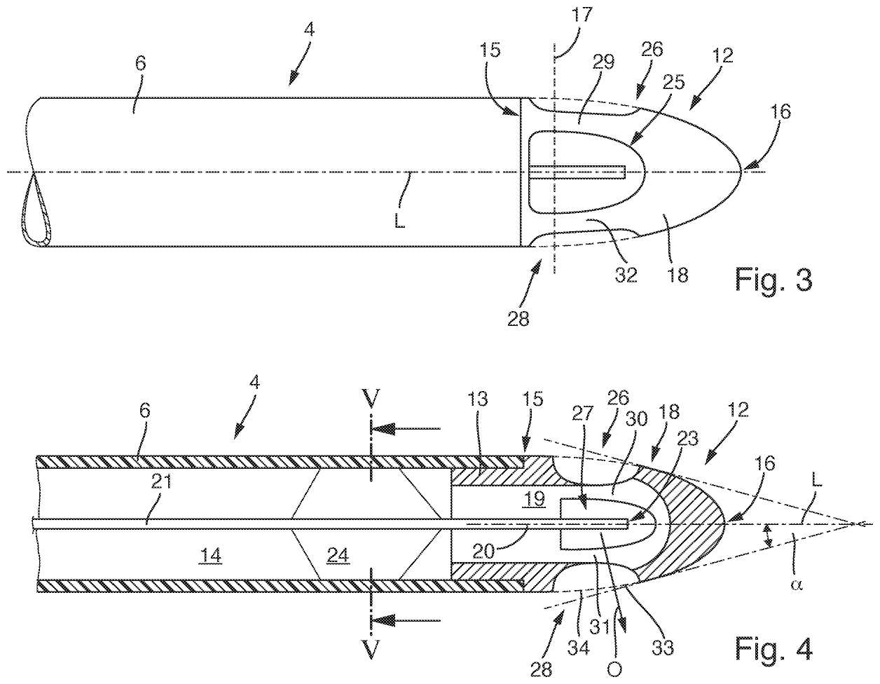

FIG. 3 illustrates a side view of an embodiment of the distal end of the FIG. 2 probe,

FIG. 4 illustrates a longitudinal view of an embodiment of the probe illustrated in FIG. 3,

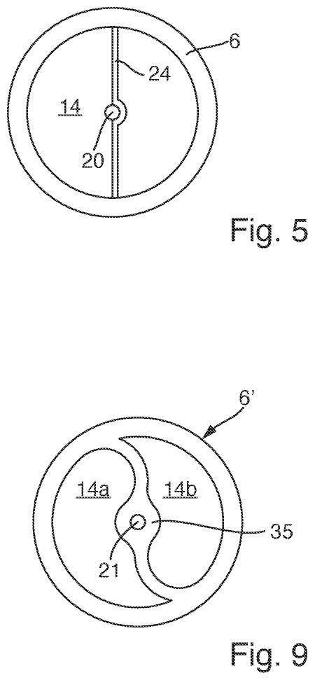

FIG. 5 illustrates the embodiment of FIG. 4 probe cut along the line V-V in FIG. 4,

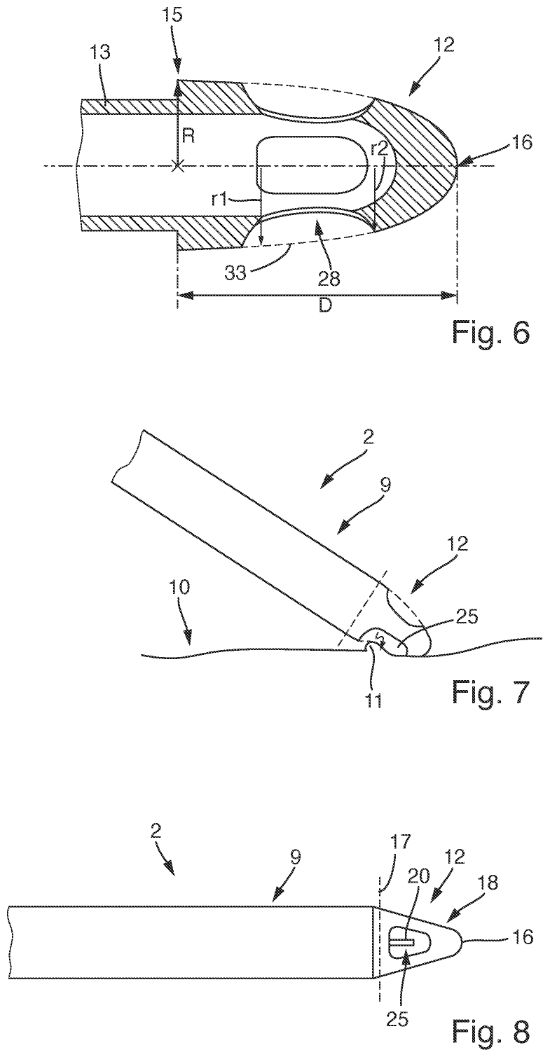

FIG. 6 illustrates an embodiment of a head piece of the probe according to FIGS. 3 and 4 in longitudinally cut illustration,

FIG. 7 illustrates an embodiment of using the probe according to FIGS. 2, 3 and 4,

FIG. 8 illustrates a modified embodiment of the probe according to the invention in schematic side view,

FIG. 9 illustrates a modified embodiment of a hose of a probe in transverse cut illustration,

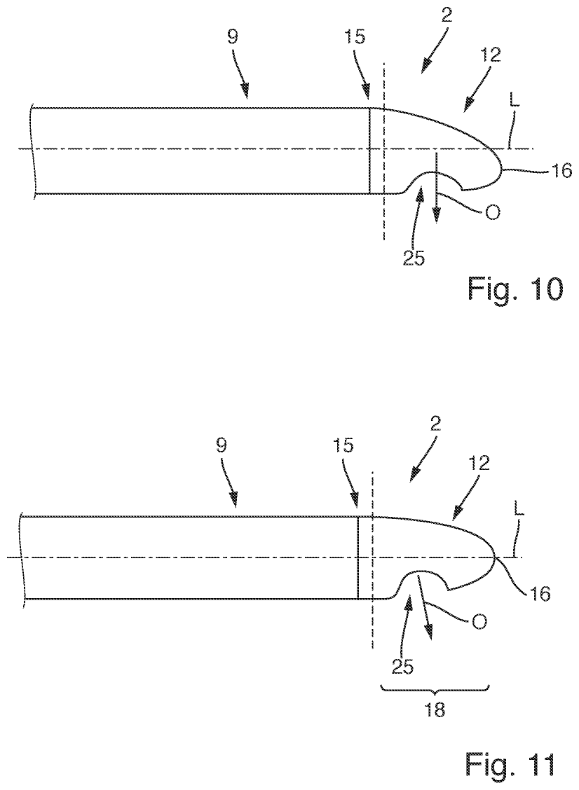

FIG. 10 illustrates an asymmetric probe according to the invention having only one plasma exit window in schematic side view, and

FIG. 11 illustrates plasma probe according to the invention having a rotationally symmetric head piece and one single lateral plasma exit window in side view.

FIG. 1 illustrates a device for plasma treatment of tissue, particularly surfaces in body lumen, having a catheter 1 and a plasma probe 2 arranged in the working channel thereof. The catheter 1 comprises a control head 3 at its proximal end by which a bending movement of its distal end 4 can be controlled. In addition, catheter 3 can be connected to an apparatus 5 that is configured, for example, for displaying an image captured at the distal end 4 and/or for supply of catheter 1 with suitable operating media, e.g. gases, flushing liquids or the like.

The plasma probe 2 comprises a hose 6, the proximal end of which can be connected and is connected to an apparatus 8 by a suitable connector 7, wherein the apparatus 8 is configured for supply of plasma probe 2 with current and gas. The apparatus comprises a gas source 8a for output of gas, e.g. inert gas, and a generator for creation of surgical current.

As apparent from FIG. 1 and particularly also FIG. 2, the plasma probe comprises a distal end 9 that can be moved out of the working channel of catheter 1 in longitudinal direction in order to treat a tissue surface 10 and particularly locations 11 in need of treatment present thereon, so-called lesions. Such locations in need of treatment can be benign—however, particularly also malign—proliferations or their preliminary stages, e.g. so-called polyps in the colon or other hollow organs.

The distal end 9 of plasma probe 2 is individually illustrated in FIGS. 3 and 4 for illustration of the specific configuration of plasma probe 2. As apparent in the open distal end of flexible hose 6 consisting preferably of plastic, a head piece 12 is inserted that projects with a tube-shaped extension 13 (FIG. 4) into the lumen 14 extending longitudinally along the entire length through hose 6. In this embodiment hose 6 is a single lumen hose without interior separation.

Preferably head piece 12 consists of an electrically insulating heat-resistant material, such as ceramic or a very temperature stable plastic. It adjoins the outer side of hose 6 at a joint 15, preferably smooth, that means without steps, and tapers up to the rounded distal tip 16 of head piece 12. The taper can already start at the joint 15 or also, as illustrated in FIG. 3, only at a certain distal distance, e.g. at a line 17 indicated in dashed manner in FIG. 3. Between the tip 16 and the dashed line 17, head piece 12 comprises an end section 18 tapering in distal direction, which can have the shape of a rounded cone. The radius and the diameter of end section 18 decreases continuously, i.e. preferably in stepless manner, in distal direction up to the distal tip 16. The surface line extending from tip 16 in direction toward joint 15 through the end section 18 can be, for example, elliptically, parabolically or can be curved otherwise.

At its tip 16 end piece 12 is closed, but configured in hollow manner otherwise and encloses an interior 19 (FIG. 4) in communication with lumen 14 and into which an electrode 20 projects.

The electrode 20 is formed by a metal pin, e.g. from stainless steel, and can be provided with a coating, e.g. of a metal, the melting point of which is lower than that of stainless steel and that is less susceptible to oxidation, such as silver. Via a line 21, electrode 20 is connected with apparatus 8, which comprises a generator for current application of electrode 20. The electrode 20 can also be formed by the distal non-insulated end of line 21. The counter-pole of electrode 20 is connected to a neutral electrode 22 (see FIG. 1), which is to be attached extensively on a suitable location of the patient for treatment of the patient.

The electrode 20 is preferably held centrally in head piece 12 and extends longitudinally and coaxially to a longitudinal center axis L of plasma probe 2. The electrode 20 can be held with its tip 23 uncovered in the interior 19 in hovering manner. Alternatively, tip 23 can be arranged engaging a pocket, which is not illustrated in FIG. 4, provided for this purpose, arranged centrally on the center axis extending in longitudinal direction L in order to remain centrally held in any case.

Additionally or alternatively, an electrode holder 24 can be provided, as illustrated in FIG. 4, which can be configured as metal sheet part. FIG. 5 illustrates the arrangement of electrode holder 24 inside lumen 14 of hose 6.

The head piece 12 comprises at least one, preferably multiple plasma exit windows 25, 26, 27, 28 that extend particularly into the tapering end section 18, which is particularly apparent from FIG. 3, or the proximal end of which is aligned with the proximal end of end section 18. For all of the embodiments applies that the electrode 20 preferably extends into the tapering end section 18 of head piece 12, whereby in turn the electrode extends along more than the half of the axial longitudinal extension of plasma exit window 25. In doing so, the plasma is created at the position of the plasma exit window and thus only little heat is transferred onto head piece 12. The plasma probe 2 is therefore particularly suitable for creation and application of extensively thermal plasma onto tissue. The plasma temperature can be above 40° C., 60° C. or 100° C. or still further above. The plasma probe is thus not only suitable for cold plasma application, but also for application of warm or hot plasma.

The plasma exit windows 25 to 28 are preferably configured identically among each other and are arranged at equal position relative to the longitudinal direction L. In so far, head piece 12 has rotational symmetry in that it can be transitioned again in a position congruent with the previous position by a rotation of 90° around the center axis extending in longitudinal direction in case of a configuration with four windows. In case of a two-window probe, a rotational symmetry of 180° is provided; a rotational symmetry of 120° is provided in case of a three-window probe.

Between windows 25 to 28 webs 29 to 32 are formed (see FIGS. 3 and 4), which preferably have a length measured in longitudinal direction L that is longer than their width measured in circumferential direction. In addition, the width of each web 29 to 32 is preferably shorter than the width of each plasma exit window 25 to 28 measured in circumferential direction. The shape of each plasma exit window 25 to 28 can be rounded. For example, the shape can be defined by a polygon, e.g. a triangle or trapezoid, having rounded corners and/or rounded edges.

Due to the at least partial arrangement of plasma exit windows 25 to 28 in the tapering end section 18 of head piece 12, the plasma exit windows 25 to 28 are orientated obliquely to the center axis L orientated in longitudinal direction. Their virtual lines 33 (FIG. 4) extending from the proximal boundary to the distal boundary include an acute angle α with the center axis. Accordingly, an opening direction O of the plasma exit window 25 to 28 extending transverse to the longitudinal direction L is inclined by an angle to the longitudinal direction L, which is less than 90°.

Also, the contour 35 of the end section shown in dashed lines in FIG. 4 decreases its radius along the extension of each opening 25 to 28. This is particularly apparent from FIG. 6 in which the radius r1 of plasma exit window 28 and radius r2 at the distal edge of plasma exit window 28 is indicated.

It is apparent from the same figure that the longitudinal head piece 12 has at least preferably a length D to be measured from the joint 15 up to the tip 16, which is longer than its radius R, particularly longer than the longest radius to be measured in cross-section. The head piece 12 thereby obtains good sliding characteristics, so that the plasma probe 2 can be moved well and easily, also into narrow body lumen.

The plasma probe 2 described so far operates with reference to FIG. 7 as follows:

During operation lumen 14 is applied with a suitable gas, e.g. an inert gas, particularly argon, by gas source 8a, so that gas flow is created along conductor 21 escaping from the plasma exit windows 25 to 28. The electrode 20 is supplied with HF-voltage by generator 8b, so that the gas is ionized and a plasma flow toward the biological tissue 10 is formed. In the ideal case the plasma flow has a flow direction that largely corresponds to the opening direction (FIG. 4).

In the embodiment according to FIG. 7, plasma exit window 25 is facing tissue 10 and the tissue area 11 in need of treatment. Due to the proximity of the area 11 in need of treatment and the electrode located behind the plasma exit window 25, an intensive plasma flow now results that particularly and predominantly impinges the location 11 in need of treatment. Concurrently, plasma probe 2 can be slidably guided on and along tissue 10. Thereby, plasma probe 2 can be guided in a flat (i.e. acute) angle relative to the surface of tissue 10 and in this manner reach all legions. Also, plasma probe 2 is suitable for narrow lumens into which it is movable well and easily, particularly because of the absence of an axial gas exit opening. Due to the small distance between electrode 20 and the tissue 10 resulting from the inclined position of the plasma exit windows 25 to 28, it can be operated with low plasma powers and thus treated carefully.

Numerous variations are possible on the plasma probe 2 described so far. FIG. 9 illustrates an embodiment modified with regard to the configuration of hose 6 having a hose 6′. The latter comprises two or more lumen 14a, 14b, which are separated from one another by separation walls. The separation walls extend from a middle section 35 to the wall of hose 6′ and thereby extend without interruption from the proximal end connector 7 to the distal end of hose 6. The middle section 35 can contain the electrical conductor 21 and concurrently serve as electrode holder. Apart therefrom, the description of the embodiment according to FIGS. 1 to 7 applies accordingly provided that the two walls separating the sublumina 14a, 14b from one another are removed in the area of the extension 13 of head piece 12. Such a hose is particularly suitable for small bending radii. The danger of buckling the lumen and hindering of the gas flow therethrough is remarkably lowered. Gas flows guided in the individual sublumina 14a, 14b, which are potentially different, are joined in head piece 12. The interior 19, particularly the portion of interior 19 present in the extension 13, can in so far form a gas collecting and balancing chamber.

FIG. 8 illustrates an embodiment of a plasma probe 2 having a head piece 12, the end section 18 of which is configured as straight cone with rounded tip 16. The plasma exit window 25 (as well as additional, for example 26 to 28) can be configured as arc-triangle-shaped windows. In addition, they can be entirely arranged in the tapering end section 18.

FIG. 10 illustrates another modified plasma probe 2 having an asymmetric head piece 12. It comprises only one single plasma exit window 25. The distal tip 16 is located away from the longitudinal center axis L. The plasma exit window 25 is arranged inside the tapering end section 18, whereby its opening direction O can be orientated in a right angle relative to the longitudinal center axis L, as illustrated in FIG. 10, or as in the other embodiments in acute angle relative to the longitudinal center axis L.

FIG. 11 illustrates in turn an embodiment of a plasma probe 2 having a head piece 12, the distal tip 16 of which is arranged centrally relative to the longitudinal center axis L. The explanations given with regard to FIGS. 1 to 9 apply accordingly for the embodiment according to FIG. 11, however, provided that only one single plasma exit window 25 is provided. The opening direction θ is orientated in an acute angle relative to the longitudinal center axis L, as already extensively explained in relation to FIG. 4. The plasma exit opening 25 can be partly or as illustrated entirely arranged in the tapering end section 18.

A flexible plasma probe comprises a head piece 12 having at least one plasma exit window 25 that is neither radially nor axially, but arranged in the tapering end section 18 of the head piece with opening direction θ orientated obliquely relative to the longitudinal center axis L. Such a plasma probe 10 simplifies and improves treatment of biological tissue, particularly lesions 11, in body lumens due to the achievable greater closeness between the electrode 20 and the lesion 11.

1. A plasma probe for treatment of biological tissue, comprising:
a flexible hose extending from a proximal end along a longitudinal center axis (L) to a distal end, the flexible hose further comprising:
a lumen connectable to a gas source,
an electrical conductor extending from the proximal end to the distal end, wherein the electrical conductor is configured to be connected to an electrical generator,
an electrode connected to the electrical conductor, and
a head piece attached to the distal end of the flexible hose, the head piece having an end section tapering in a distal direction,
wherein at least one plasma exit window is configured in the head piece, the plasma exit window is at least partly arranged in the tapering end section.

2. The plasma probe according to claim 1, wherein the head piece is made of a heat-resistant electrically insulating material.

3. The plasma probe according to claim 1, wherein the plasma exit window is orientated transverse to the longitudinal center axis (L).

4. The plasma probe according to claim 1, wherein the head piece further comprises multiple plasma exit windows distanced from one another in circumferential direction at the same axial position, which are at least partly arranged in the tapering end section.

5. The plasma probe according to claim 1, wherein the tapering end section is rounded.

6. The plasma probe according to claim 1, wherein the tapering end section is longitudinally rounded.

7. The plasma probe according to claim 1, wherein the head piece has a circular cross-section having a radius (R) at its hose side end, which is smaller than a length (D) measured from the hose side end to a tip of the head piece.

8. The plasma probe according to claim 1, wherein the electrode is fixated in the hose.

9. The plasma probe according to claim 1, wherein the electrode is fixated in the head piece.

10. The plasma probe according to claim 1, wherein the electrode comprises a distal end that does not contact the head piece.

11. The plasma probe according to claim 1, wherein the hose comprises one single gas conveying lumen.

12. The plasma probe according to claim 1, wherein the hose comprises multiple gas conveying lumens.

13. The plasma probe according to claim 1, wherein the head piece comprises only one single plasma exit window.

14. The plasma probe according to claim 1, wherein the tapering section of the head piece is figured rotationally symmetrically relative to the longitudinal center axis (L).

15. The plasma probe according to claim 1, wherein the tapering section of the head piece is asymmetrically configured relative to the longitudinal center axis (L).
